(19) United States
(12) Patent Application Publication
(10) Pub. No.: US 2026/0335619 A1
(43) Pub. Date: Oct. 29, 2026

(54) MEDICAL TUBE ADAPTER

(71) Applicant: Qidia Solutions Inc., Staten Island, NY (US)

(72) Inventors: Cyndia Alvarado, Staten Island, NY (US); Raqquyah Webb, Brooklyn, NY (US); Edwin Chan, Brooklyn, NY (US)

(21) Appl. No.: 19/658,294

(22) Filed: Apr. 24, 2026

Related U.S. Application Data
(60) Provisional application No. 63/796,689, filed on Apr. 29, 2025.

(51) Int. Cl. A61M 39/10

(52) U.S. Cl. CPC A61M 39/10; A61M 2039/1077

(57) ABSTRACT

A medical tube adapter includes a first port and a second port. The first port is configured to connect to at least three different sizes of tubing.

### RELATED APPLICATIONS

This application claims benefit of U.S. Provisional Application No. 63/796,689, filed Apr. 29, 2025, the contents of which are incorporated by reference in their entirety.

### TECHNICAL FIELD

The present disclosure relates to adapters, and more specifically, to a medical tube adapter.

### BRIEF DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates a medical tube adapter including two ports, according to some embodiments.

FIG. 2 illustrates a medical tube adapter including three ports, according to some embodiments.

FIGS. 3A-F illustrate a medical tube adapter including two ports and an extendable sizing adapter, according to some embodiments.

FIGS. 4A-F illustrate a medical tube adapter including three ports and an extendable sizing adapter, according to some embodiments.

FIGS. 5A-8B illustrate a connectable medical tube adapter including various adapters, according to some embodiments.

FIG. 9 illustrates a one-way valve adapter, according to some embodiments.

FIG. 10 illustrates a sensor adapter, according to some embodiments.

FIGS. 11A-D illustrate medical tube adapters, according to some embodiments.

FIG. 12 illustrates a medical tube adapter, according to some embodiments.

FIG. 13 illustrates a medical tube adapter, according to some embodiments.

### DETAILED DESCRIPTION OF EMBODIMENTS

Embodiments described herein are related to a medical tube adapter.

Hoses and tubing are used to move fluids (e.g., substances this flow, such as a liquid or a gas) from one place to another. Hoses and tubing may not be sufficiently long or short enough to satisfy an individual purpose. Hoses, tubing, and medical equipment are of different sizes. Fluids from multiple hoses and/or tubing may be combined into a single hose and/or tube. Fluids from a single hose and/or tube may be divided into multiple hoses and/or tubes. Different hoses and tubing may not directly connect to other components (e.g., medical equipment). Adapters are often used to link hoses, tubing, and/or other components (e.g., medical equipment, etc.) together. Often, hoses, tubing, and/or other components (e.g., medical equipment, etc.) are to be connected quickly as different uses arise (e.g., a patient needs one or more new fluids and/or medical equipment). Often, the correct adapters are not readily accessible for a particular configuration of particular sizes and quantities of hoses, tubes, and/or other components. Issues arise when trying to connect hoses, tubing, and/or medical equipment together that are not the same size, or when trying to introduce a new fluid or pathway into hoses or tubing.

One example where such issues are seen is in a hospital or other medical setting. Medical professionals may need to attach various hoses, tubes, and/or other components quickly and efficiently (e.g., medical equipment) carrying various fluids to a patient. In emergency situations, the moments it takes to attempt to find the correct adapter and/or create a makeshift solution to make these attachments and connections can be the difference between successful and unsuccessful patient care, and extended delays could result in a patient’s death.

Conventionally, adapters used in medical settings are fixed in size, meaning that the adapter is configured to connect a specific size of tubing to another specific size of tubing. If either tubing is a different size than required by the adapter, another adapter must be found. This could cause delays in patient care, leading to health risks or complications.

In some instances, the configuration needed to connect two or more hoses, tubes, and/or other components (e.g., medical equipment) may not exist or may not be readily available, which leads to fitting multiple adapters together to attempt to arrive at the proper configuration. As more adapters, hoses, and/or tubes are fitted together, more points of weakness are introduced. This could result in the connecting components falling apart, leading to a reduction in patient care and increased health risks.

Conventional adapter solutions may also be stored in various locations. For instance, if multiple components are to be used to connect different hoses and/or tubes, the multiple components may be in different drawers, cupboards, different parts of the medical facility, or may need to be purchased. Finding the correct components takes time, which reduces efficiency in providing patient care and could result in increased health risks.

The devices, systems, and methods of the medical tube adapter disclosed in this application overcome at least the challenges mentioned above.

A medical tube adapter may include a first port and/or a second port. The first port may be configured to connect to at least three different sizes of tubing. In some embodiments, the second port is configured to connect to at least three different sizes of tubing. The respective different sizes of tubing for the first port and the second port may be the same respective different sizes, or different respective different sizes. In some embodiments, the medical tube adapter further includes a third port. The third port may be configured to connect to at least two different sizes of tubing, which may be the same or different from the different sizes of tubing of the first port and/or the second port.

The present disclosure has advantages over conventional solutions. The present disclosure may provide an adapter configured to quickly attach various types and sizes of hoses, tubes, and/or other components without the use of multiple components to provide increased efficiency and speed in patient care. The present disclosure may provide an adapter with flexible sizing (e.g., not fixed in size), allowing for multiple configurations to connect a variety of tubing sizes to reduce delays in patient care. The present disclosure may connect various components of tubing using a single device, rather than requiring multiple components. The present disclosure may provide an adapter with multiple sizing options contained in a single unit, which reduces the amount of time it takes to find the component to be used, increases efficiency in providing patient care, and reduces health risks. The present disclosure may increase efficiency by having the functionality of various adapters in a single unit.

The present disclosure may be used to connect two or more hoses, tubes, medical components, adapters, conduits, lines, channels, sleeves, etc. Although some embodiments of the present disclosure discuss a medical tube adapter to connect multiple components of medical tubing together, in some embodiments, the device of the present disclosure may be used to connect (e.g., couple, join, attach, engage, interface, link, align, etc.) other types of conduits unrelated to medical care or treatment.

Although some embodiments of the present disclosure are discussed with regards to medical tube adapters used in medical environments, in some embodiments, the present disclosure can be applied to other environments (e.g., plumbing, dentistry, heating ventilation and air conditioning (HVAC), etc.) and/or connecting other types of components.

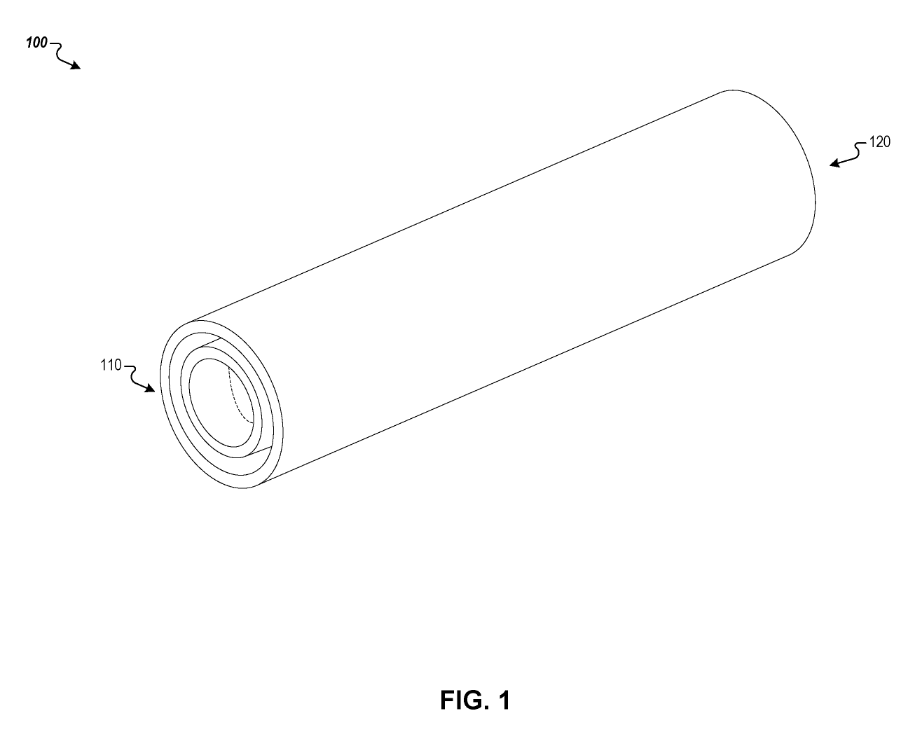

FIG. 1 illustrates a medical tube adapter 100 including two ports 110 and 120, according to some embodiments. In some embodiments, the medical tube adapter 100 is between approximately 2 and 6 inches in length. In some embodiments, the medical tube adapter 100 is between 0.5 and 3 inches in height. A first port 110 may be located on a first distal end of the medical tube adapter 100, and a second port 120 may be located on a second distal end of the medical tube adapter 100.

In some embodiments, the medical tube adapter 100 is made of a hard material configured to maintain a fixed shape and size. The material may be one of polyvinyl chloride (PVC), polyurethane, thermoplastic elastomers (TPE), polyethylene (PE), polypropylene (PP), stainless steel, metal, ceramic, rubber, silicone, or another similar material.

The first port 110 and/or the second port 120 may have diameter (e.g., an outer diameter) of between 10 millimeter (mm) and 30 mm. In some embodiments, the first port 110 and/or the second port 120 is configured to connect to at least a 15 mm tube and/or a 22 mm tube. In some embodiments, the first port 110 is configured to connect to different sizes of tubing from the second port 120. In some embodiments, the first port 110 is configured to connect to a same size of tubing as the second port 120.

In some embodiments, the first port 110 has an inner interface (e.g., an inner cylindrical lip) and an outer interface (e.g., an outer cylindrical lip). The inner interface and the outer interface may be concentric to each other. In some embodiments, the first port 110 includes more than one inner interface concentric with the other interfaces. A hose, tube, and/or other component (e.g., medical equipment, adapters, etc.) may have a friction fit (e.g., held together by tight contact and resistance between surfaces without the use of threads, clamps, or adhesives) to an outer surface of the outer interface, to an inner surface of the outer interface, to an outer surface of the inner interface, and/or to an inner surface of the inner interface, providing one port configured to connect to four different sizes of hoses, tubes, and/or other components.

Although the second port 120 is not shown in FIG. 1, the second port 120 may be substantially similar in structure to the first port 110. In some embodiments, the second port 120 has a structure different from that of the first port 110. For example, in some embodiments, the second port 120 has one interface, where a hose, tube, and/or other component has a friction fit to an outer surface of the interface and/or to an inner surface of the interface, providing one port configured to connect to two different sizes of hoses, tubes, and/or other components. In another example, the second port 120 may have multiple interfaces concentric to each other that are the same size as those found on the first port 110. In yet another example, the second port 120 may have multiple interfaces concentric to each other with at least one interface having a size different from that of the interfaces of the first port 110. The first port 110 and/or the second port 120 may have any number of interfaces (e.g., one interface, two interfaces, three interfaces, etc.) that are concentric to each other. In some embodiments, each interface of the first port 110 and/or the second port 120 has an inner surface and an outer surface.

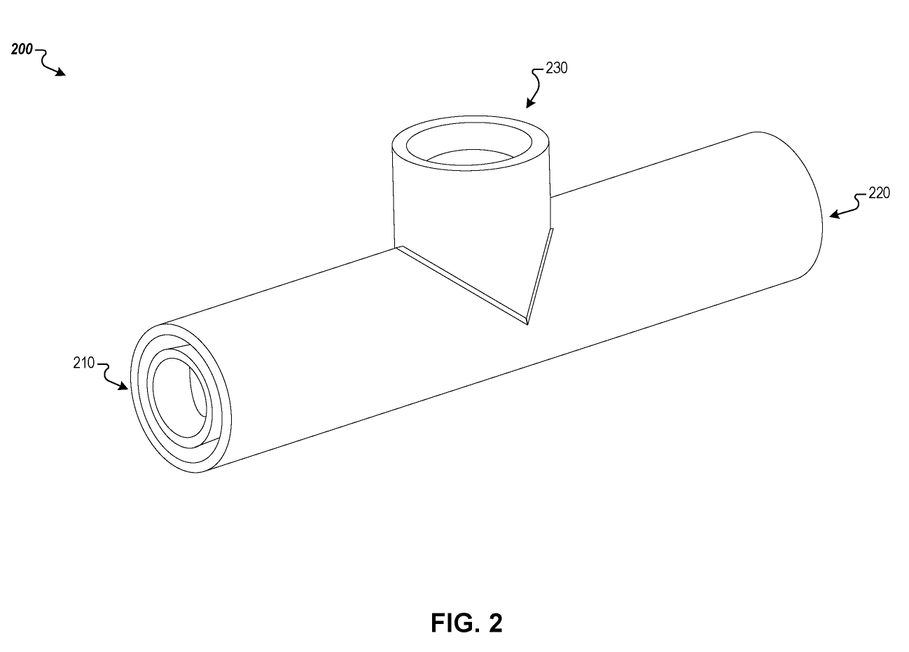

FIG. 2 illustrates a medical tube adapter 200 including three ports 210, 220, and 230, according to some embodiments. The medical tube adapter 200 may be composed of a material as described in relation to the medical tube adapter 100 of FIG. 1. In some embodiments, the medical tube adapter 200 is between approximately 2 and 6 inches in length. In some embodiments, the medical tube adapter 100 is between 0.5 and 6 inches in height. A first port 210 may be located on a first distal end of the medical tube adapter 200, and a second port 220 may be located on a second distal end of the medical tube adapter 200. A third port 230 may be located between the first port 210 and the second port 220. In some embodiments, the third port 230 is substantially perpendicular to the first port 210 and/or the second port 220. In some embodiments, the third port 230 is positioned at a midpoint of the medical tube adapter 200 between the first port 210 and the second port 220. The medical tube adapter 200 may be substantially similar to the medical tube adapter 100 of FIG. 1, with the addition of the third port 230.

In some embodiments, the first port 210 is substantially similar to the first port 110 of FIG. 1 and has an inner interface (e.g., an inner cylindrical lip) and an outer interface (e.g., an outer cylindrical lip). The inner interface and the outer interface may be concentric to each other. In some embodiments, the first port 210 includes more than one inner interface concentric with the other interfaces. A hose, tube, and/or other component (e.g., medical equipment, adapters, etc.) may have a friction fit (e.g., held together by tight contact and resistance between surfaces without the use of threads, clamps, or adhesives) to an outer surface of the outer interface, to an inner surface of the outer interface, to an outer surface of the inner interface, and/or to an inner surface of the inner interface, providing one port configured to connect to four different sizes of hoses, tubes, and/or other components.

Although the second port 220 is not shown in FIG. 2, the second port 220 may be substantially similar in structure to the first port 210. In some embodiments, the second port 220 has a structure different from that of the first port 210. For example, in some embodiments, the second port 220 has one interface, where a hose, tube, and/or other component has a friction fit to an outer surface of the interface and/or to an inner surface of the interface, providing one port configured to connect to two different sizes of hoses, tubes, and/or other components. In another example, the second port 220 may have multiple interfaces concentric to each other that are the same size as those found on the first port 110. In yet another example, the second port 120 may have multiple interfaces concentric to each other with at least one interface having a size different from that of the interfaces of the first port 110. The first port 110 and/or the second port 120 may have any number of interfaces (e.g., one interface, two interfaces, three interfaces, etc.) that are concentric to each other. In some embodiments, each interface of the first port 110 and/or the second port 120 has an inner surface and an outer surface.

The third port 230 may be substantially similar to the first port 210 and/or the second port 220. In some embodiments, the third port 230 has one interface, where a hose, tube, and/or other component has a friction fit to an outer surface of the interface and/or to an inner surface of the interface, providing one port configured to connect to two different sizes of hoses, tubes, and/or other components. In some embodiments, the third port 230 has multiple interfaces concentric with each other which are configured to connect to more than two difference sizes of hoses, tubes, and/or other components.

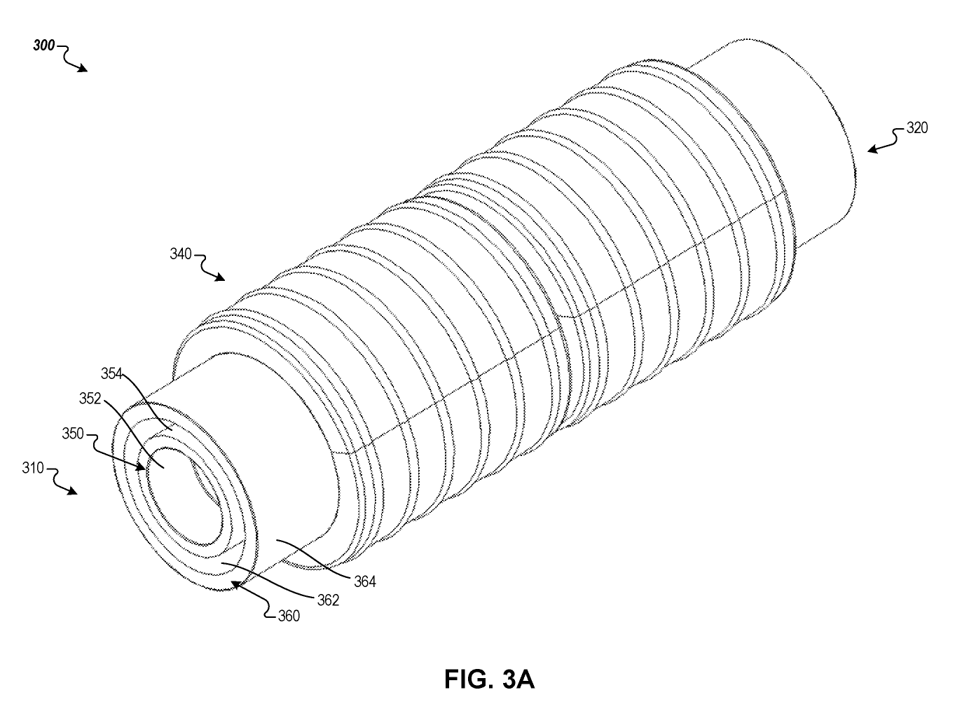

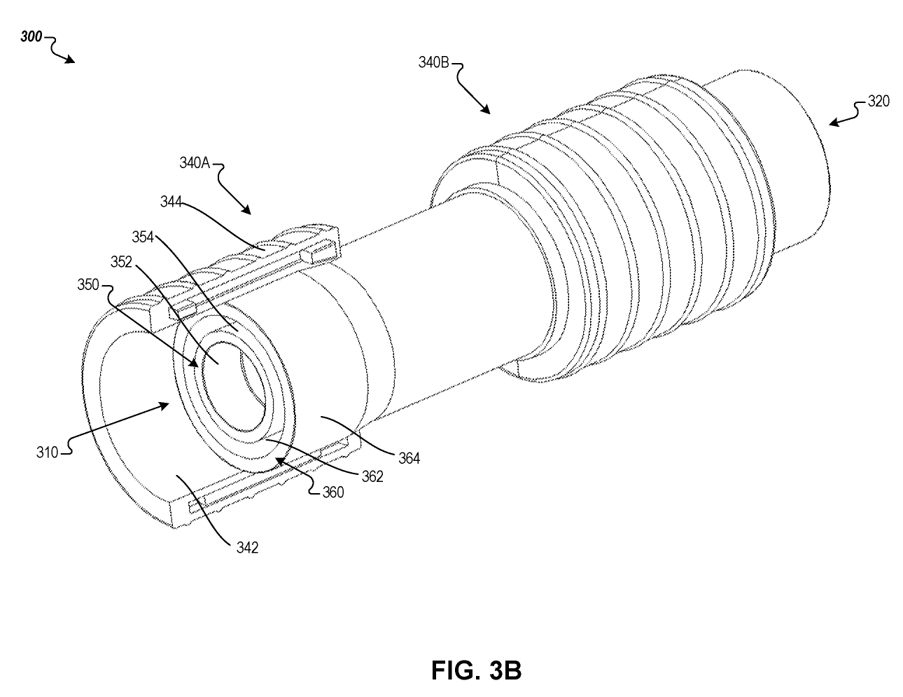

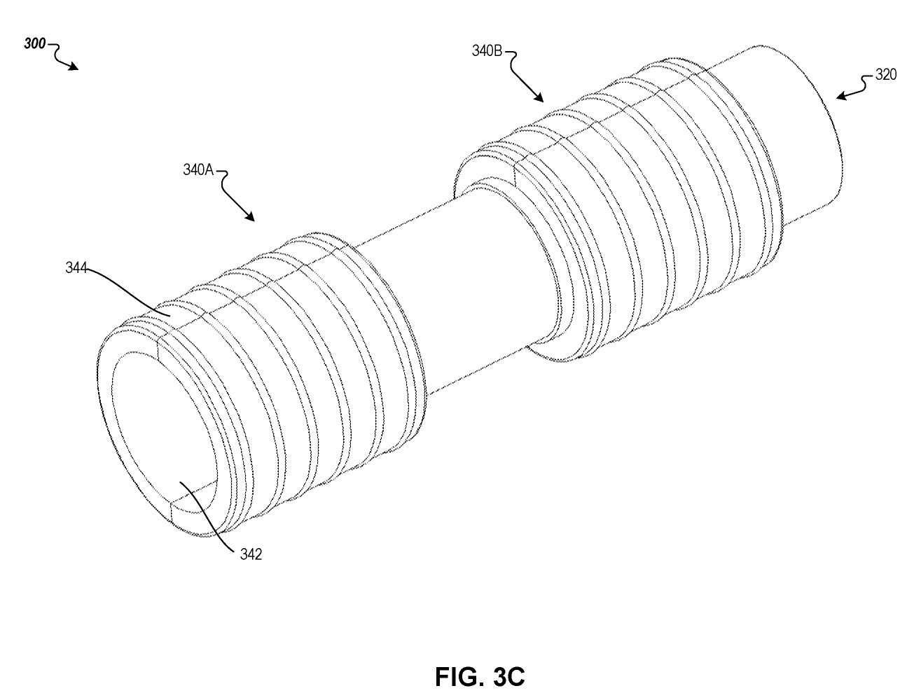

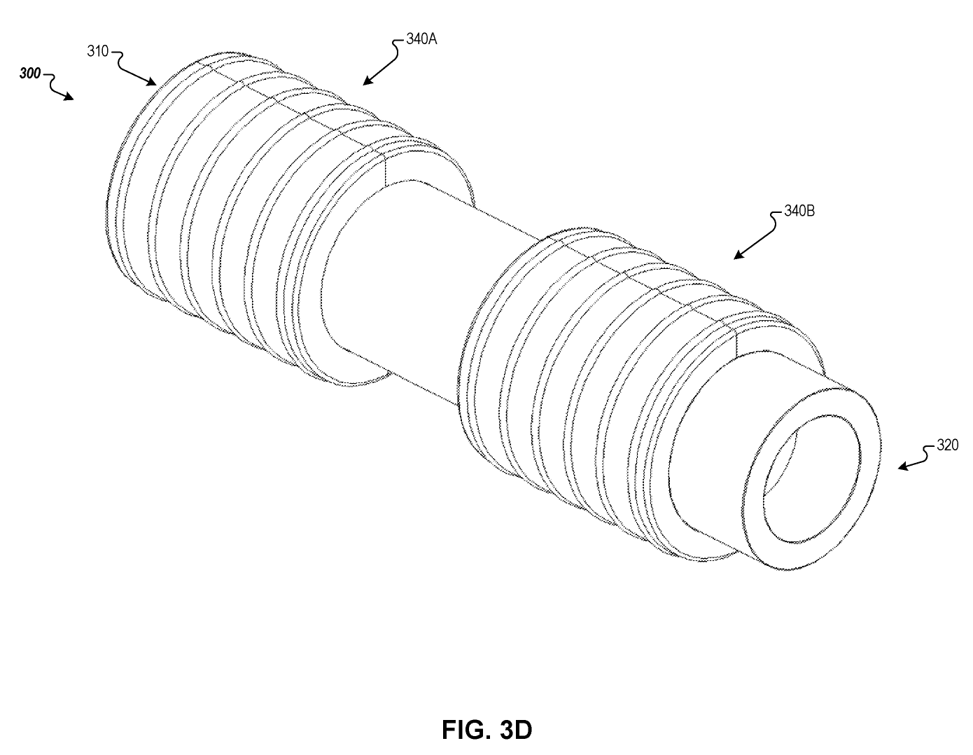

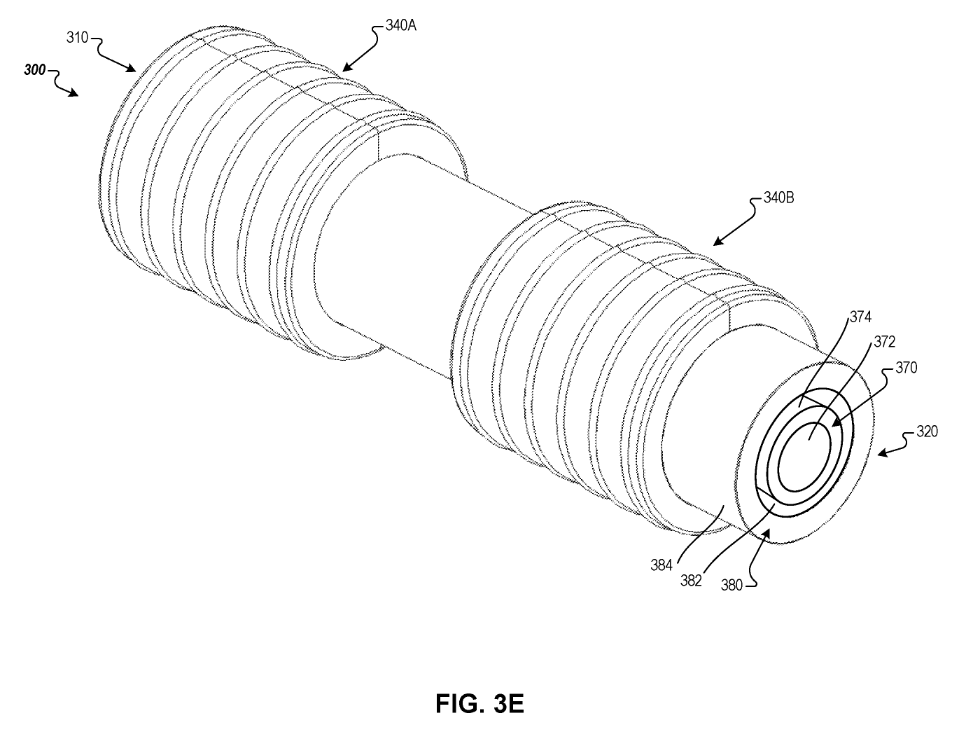

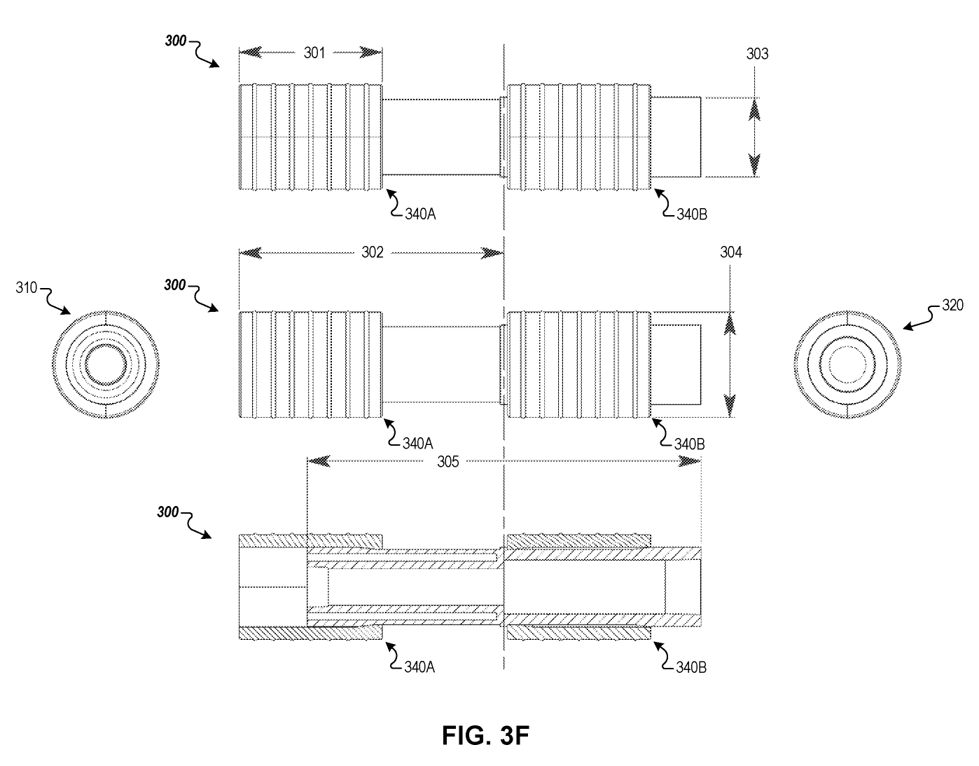

FIGS. 3A-F illustrate a medical tube adapter 300 including two ports 310 and 320 and an extendable sizing adapter 340, according to some embodiments. FIG. 3A illustrates a perspective view of the medical tube adapter 300 with the extendable sizing adapter 340 in a retracted position. FIG. 3B illustrates a perspective view of the medical tube adapter 300 with a partial cross-section of a first extendable sizing adapter portion 340A in an extended position. FIG. 3C illustrates a perspective view of the medical tube adapter 300 with the first extendable sizing adapter portion 340A in an extended position. FIGS. 3A-C illustrate perspective views with the first port 310 in view. FIGS. 3D-E illustrate perspective views of the medical tube adapter 300 with the first extendable sizing adapter portion 340A in an extend position with a second port 320 in view. FIG. 3D illustrates the second port 320 with a cylindrical lip. FIG. 3E illustrates the second port 320 with an inner cylindrical lip and an outer cylindrical lip. FIG. 3F illustrates various dimensions of the medical tube adapter 300, according to some embodiments. Components and features of FIGS. 1, 2, 3A, 3B, 3C, 3D, 3E, and/or 3F that have the same or similar name and/or reference number may have the same or similar functionality, material, components, etc.

FIG. 3A illustrates a perspective view of the medical tube adapter 300 including the extendable sizing adapter 340 in a retracted position, according to some embodiments. A retracted position may be a position in which the extendable sizing adapter 340 is substantially centered on the medical tube adapter 300 and is withdrawn from a first distal end (e.g., a first port 310) and/or a second distal end (e.g., a second port 320) of the medical tube adapter 300. In some embodiments, the extendable sizing adapter 340 includes more than one portion of an extendable sizing adapter, as seen in FIG. 3B. In some embodiments, the extendable sizing adapter 340 is configured to slidably interface with the medical tube adapter 300 (e.g., the extendable sizing adapter 340 can slide along the medical tube adapter 300) between the retracted position and an extended position. An extended position may be a position in which the extendable sizing adapter 340 is disposed past the distal ends of the medical tube adapter 300.

The medical tube adapter 300 may be tapered toward the center portion, with the diameter of the medical tube adapter 300 increasing toward a first distal portion that includes the first port 310 and increasing towards a second distal portion that includes the second port 320. In some embodiments, when the extendable sizing adapter 340 is in the retracted position, the increased diameter prevents the extendable sizing adapter 340 from sliding forward into the extended position without external force. In some embodiments, when the extendable sizing adapter 340 is in the extended position, the increased diameter prevents the extendable sizing adapter 340 from sliding back into the retracted position without external force.

In some embodiments, the extendable sizing adapter 340 is a sheath, a sleeve, an enclosure, a housing, a case, and/or an outer jacket. The extendable sizing adapter 340 may be a tube with an inner surface and an outer surface, where the inner surface is configured to attach to the medical tube adapter 300. The extendable sizing adapter 340 may have an inner diameter and/or inner circumference (e.g., measured from the inner surface) that is substantially similar to an outer diameter and/or outer circumference of the first port 310 and/or the second port 320 (e.g., measured from an outer surface of the first port 310 and/or the second port 320). In some embodiments, the extendable sizing adapter 340 having an inner diameter and/or inner circumference that is substantially similar to an outer diameter and/or outer circumference of the first port 310 and/or the second port 320 enables a substantially airtight seal between the first port 310 and/or the second port 320 and a hose, tube, and/or other component with a diameter substantially similar to the diameter of the first port 310 and/or the second port 320 when the extendable sizing adapter 340 is in the extended position.

In some embodiments, the extendable sizing adapter 340 is a locking extendable sizing adapter, where the locking extendable sizing adapter is configured to lock in place relative to the medical tube adapter 300 and lock a connected hose, tube, and/or other component in place (e.g., aligned with the first port 310 and/or the second port 320).

The medical tube adapter 300 may further include the first port 310 and the second port 320. The first port 310 may be configured to connect to at least three different sizes of tubing. In some embodiments, the first port 310 is configured to connect to at least four different sizes of tubing. In some embodiments, the first port 310 includes an inner cylindrical lip 350 and an outer cylindrical lip 360. The inner cylindrical lip 350 may have an outer diameter of between 10 mm and 20 mm. In some embodiments, the inner cylindrical lip 350 is configured to connect to a 15 mm tubing. In some embodiments, the inner cylindrical lip 350 is configured to connect to other sizes of tubing.

The inner cylindrical lip 350 may include a first inner surface 352 and a first outer surface 354. The first inner surface 352 may be configured to connect with a first size of tubing. In some embodiments, the first inner surface 352 is configured to connect with tubing and/or adapters smaller than a 15 mm tubing. The first outer surface 354 may be configured to connect with a second size of tubing. In some embodiments, the first outer surface 354 is configured to connect with tubing and/or adapters of a same or similar diameter as a 15 mm tubing.

The inner cylindrical lip 350 may be concentrically located within the outer cylindrical lip 360. The outer cylindrical lip 360 may include a second inner surface 362 and a second outer surface 364. The second inner surface 362 may be configured to connect with a third size of tubing. In some embodiments, the second inner surface 362 is configured to connect with tubing and/or adapters slightly larger than the tubing and/or adapters that may connect with the first outer surface 354. The second outer surface 364 may be configured to connect with a fourth size of tubing. In some embodiments, the second outer surface 364 is configured to connect with tubing and/or adapters of a same or similar diameter as a 22 mm tubing. In some embodiments, the fourth size of tubing has a diameter larger than the third size of tubing, which has a diameter larger than the second size of tubing, which has a diameter larger than the first size of tubing.

FIGS. 3B-C illustrate perspective views of the medical tube adapter 300 including a first extendable sizing adapter portion 340A in the extended position, according to some embodiments. FIG. 3B illustrates the first extendable sizing adapter portion 340A as a partial cross-section. FIG. 3C illustrates the first extendable sizing adapter portion 340A intact. As illustrated, the extendable sizing adapter 340 includes the first extendable sizing adapter portion 340A and a second extendable sizing adapter portion 340B. In some embodiments, the first extendable sizing adapter portion 340A is configured to slide toward and extend beyond the first port 310. In some embodiments, the second extendable sizing adapter portion 340B is configured to slide toward and extend beyond the second port 320. The first extendable sizing adapter portion 340A and the second extendable sizing adapter portion 340B may be configured to slide away from each other in opposite directions parallel to the medical tube adapter 300.

The first extendable sizing adapter portion 340A may include an extendable sizing adapter inner surface 342 and an extendable sizing adapter outer surface 344. The extendable sizing adapter inner surface 342 may be configured to connect to tubing and/or an adapter with an outer diameter substantially similar to the outer diameter of the outer cylindrical lip 360. The extendable sizing adapter outer surface 344 may be configured to connect to tubing and/or an adapter with an outer diameter larger than the outer cylindrical lip 360.

The second extendable sizing adapter portion 340B may have the same or similar features as described in relation to the first extendable sizing adapter portion 340A.

FIGS. 3D-E illustrate perspective views of the medical tube adapter 300 including a second port, according to some embodiments. FIG. 3D illustrates the second port 320 with a cylindrical lip. FIG. 3E illustrates the second port 320 with an inner cylindrical lip 370 and an outer cylindrical lip 380. In some embodiments (e.g., as illustrated in FIG. 3D), the second port 320 is configured to connect with two different sizes of tubing (e.g., one size that fits within the cylindrical lip of the second port 320, and one size that fits around the circumference of the cylindrical lip of the second port 320). In some embodiments (e.g., as illustrated in FIG. 3E), the second port 320 includes an inner cylindrical lip 370 and an outer cylindrical lip 380. With this configuration, the second port 320 is configured to connect with at least three different sizes of tubing and is similar to the structure described in relation to the first port 310. In some embodiments, the second port 320 is configured to connect to additional sizes of tubing when the second extendable sizing adapter portion 340B is in the extended position.

FIG. 3F illustrates various dimensions of the medical tube adapter 300, according to some embodiments. The medical tube adapter 300 may have an adapter body length 305 of about 75 mm to about 145 mm.

The first extendable sizing adapter portion 340A and the second extendable sizing adapter portion 340B may each have an extendable sizing adapter portion length 301. The extendable sizing adapter portion length 301 may be about 20 mm to about 60 mm. In some embodiments, the extendable sizing adapter portion length 301 of the first extendable sizing adapter portion 340A is substantially similar to the extendable sizing adapter portion length of the second extendable sizing adapter portion 340B. In some embodiments, the extendable sizing adapter portion length 301 of the first extendable sizing adapter portion 340A is different from the extendable sizing adapter portion length of the second extendable sizing adapter portion 340B. In some embodiments, the combined length of the extendable sizing adapter portion length 301 of the first extendable sizing adapter portion 340A and the extendable sizing adapter portion length of the second extendable sizing adapter portion 340B does not exceed the adapter body length 305.

In some embodiments, the first extendable sizing adapter portion 340A is confined to a first portion of the medical tube adapter having an adapter body first portion length of approximately half of the adapter body length 305. In some embodiments, a first extended body length 302 corresponds to the first extendable sizing adapter portion 340A being in an extended position. The first extended body length 302 may be about 60 mm to about 90 mm. In some embodiments, the second extendable sizing adapter portion 340B is confined to a second portion of the medical tube adapter having an adapter body first portion length of approximately half of the adapter body length 305. In some embodiments, a second extended body length corresponds to the second extendable sizing adapter portion 340B being in an extended position. The second extended body length may be substantially similar to the first extended body length 302.

The second port 320 may have a port height 303, which is a diameter of the second port 320 from one or more outer edges of the second port 320. In some embodiments, the port height 303 is about 18 mm to about 26 mm. The first port 310 may have a port height substantially similar to the port height 303. In some embodiments, the first port 310 has a port height different from the port height 303.

The second extendable sizing adapter portion 340B may have an adapter height 304, which is a diameter of the second extendable sizing adapter portion 340B from one or more outer edges of the second extendable sizing adapter portion 340B. In some embodiments, the adapter height 304 is about 25 mm to about 33 mm. The first extendable sizing adapter portion 340A may have an adapter height substantially similar to the adapter height 304. In some embodiments, the first extendable sizing adapter portion 340A has an adapter height different from the adapter height 304.

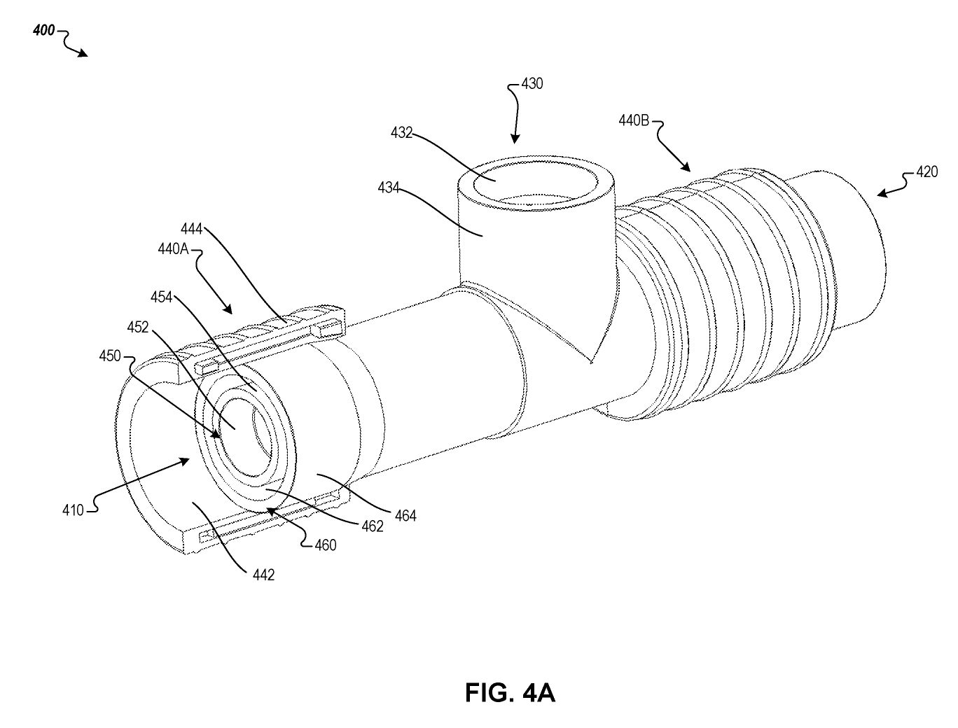

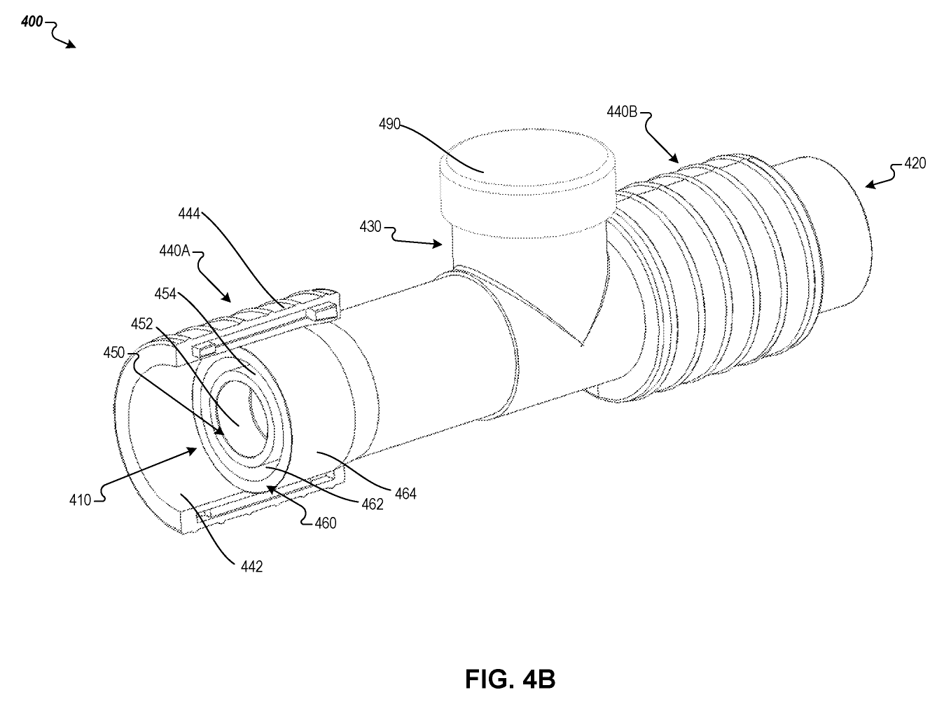

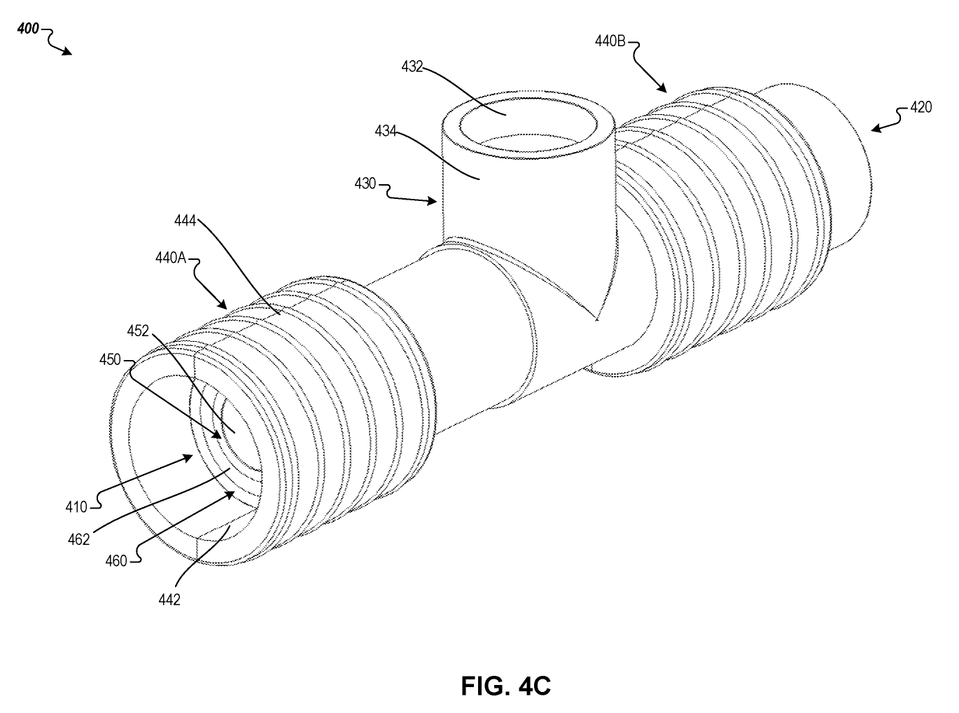

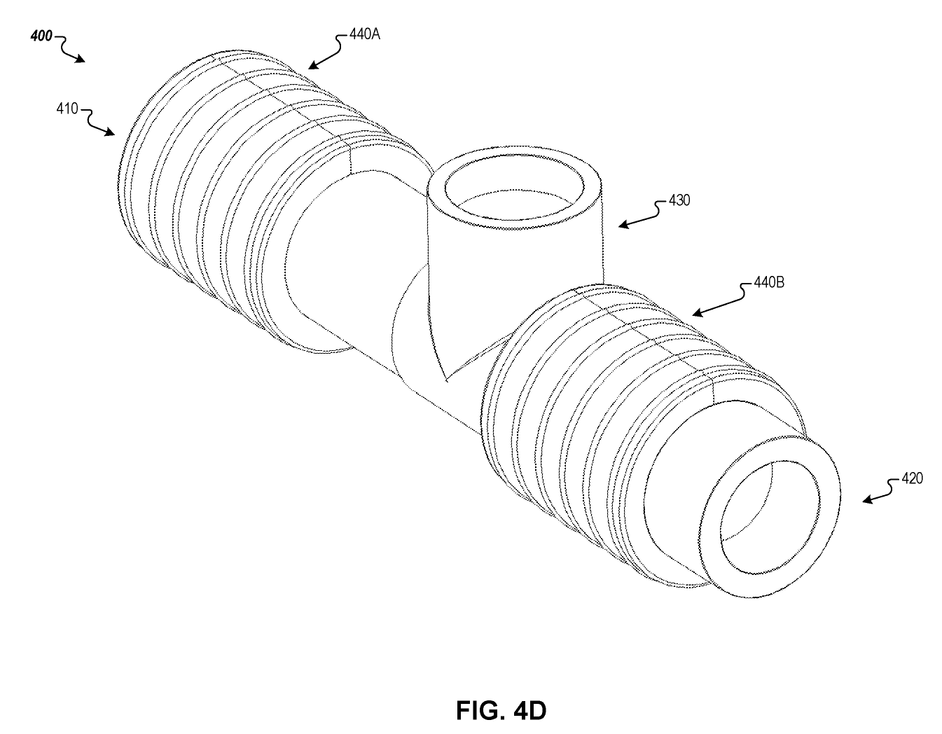

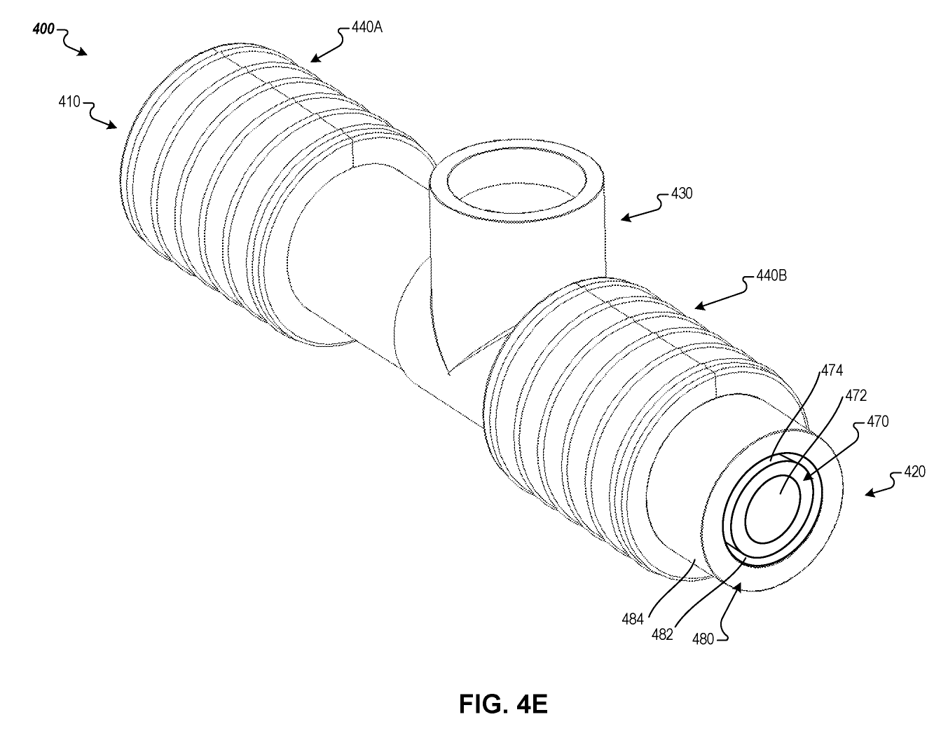

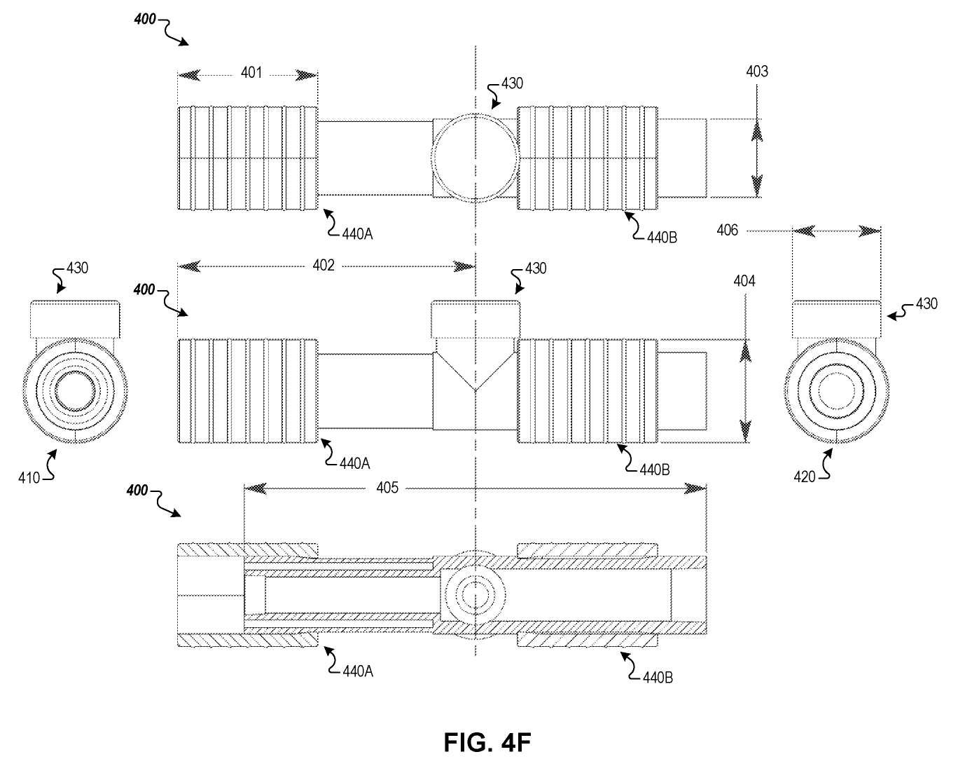

FIGS. 4A-F illustrate a medical tube adapter 400 including three ports 410, 420, and 430 and an extendable sizing adapter 440, according to some embodiments. FIG. 4A illustrates a perspective view of the medical tube adapter 400 with the first extendable sizing adapter portion 440A and the second extendable sizing adapter portion 440B in a retracted position. FIG. 4B illustrates a perspective view of the medical tube adapter 400 with a partial cross-section of the first extendable sizing adapter portion 440A in an extended position. FIG. 4C illustrates a perspective view of the medical tube adapter 400 with the first extendable sizing adapter portion 440A in an extended position. FIGS. 4A-C illustrate perspective views with the first port 410 in view. FIGS. 4D-E illustrate perspective views of the medical tube adapter 400 with the first extendable sizing adapter portion 440A in an extend position with a second port 420 in view. FIG. 4D illustrates the second port 420 with a cylindrical lip. FIG. 4E illustrates the second port 420 with an inner cylindrical lip 470 and an outer cylindrical lip 480. FIG. 4F illustrates various dimensions of the medical tube adapter 400, according to some embodiments. Components and features of FIGS. 1, 2, 3A, 3B, 3C, 3D, 3E, 4A, 4B, 4C, 4D, 4E, and/or 4F that have the same or similar name and/or reference number may have the same or similar functionality, material, components, etc.

FIG. 4A illustrates a perspective view of the medical tube adapter 400 with the first extendable sizing adapter portion 440A and the second extendable sizing adapter portion 440B in a retracted position, according to some embodiments. The medical tube adapter 400, first extendable sizing adapter portion 440A, and second extendable sizing adapter portion 440B are substantially similar to the components described in FIGS. 3A-E, with the addition of a third port 430. In some embodiments, the third port 430 is configured to connect to at least two different sizes (e.g., a fifth size and/or a sixth size) of tubing and/or adapters (e.g., one size that fits within the third port 430, and one size that fits around the circumference of the third port 430).

In some embodiments, the third port 430 is used to introduce a second fluid (e.g., gas, oxygen, medicine, water, etc.) to a first fluid transmitting between the first port 410 and the second port 420. The third port 430 may be configured to receive an adapter configured to measure the flow of the fluid (e.g., oxygen flow, nitric oxide flow, carbon dioxide flow, etc.). The third port 430 may be configured to receive an adapter configured to evaluate the amount of fluid going to a patient.

In some embodiments (e.g., as seen in FIG. 4B), the third port 430 is configured to receive a cover 490. The cover 490 may be of a substantially similar material as the rest of the medical tube adapter 400 or may be a different material. In some embodiments, the cover 490 may enclose a portion of the third port 430 such that any fluid passing within the medical tube adapter 400 does not escape. For example, if the medical tube adapter 400 were used to connect two hoses, tubes, and/or other components, the third port 430 could be enclosed with the cover 490 to prevent fluid from escaping from the third port 430. In some embodiments, the cover 490 includes a gasket to further prevent fluid from escaping when enclosing the third port 430. In some embodiments, the cover 490 is an airtight cover to completely prevent fluid from escaping when enclosing the third port 430.

FIG. 4F illustrates various dimensions of the medical tube adapter 400, according to some embodiments. The medical tube adapter 400 may have an adapter body length 405 of about 95 mm to about 170 mm.

The first extendable sizing adapter portion 440A and the second extendable sizing adapter portion 440B may each have an extendable sizing adapter portion length 401. The extendable sizing adapter portion length 401 may be about 20 mm to about 60 mm. In some embodiments, the extendable sizing adapter portion length 401 of the first extendable sizing adapter portion 440A is substantially similar to the extendable sizing adapter portion length of the second extendable sizing adapter portion 440B. In some embodiments, the extendable sizing adapter portion length 401 of the first extendable sizing adapter portion 440A is different from the extendable sizing adapter portion length of the second extendable sizing adapter portion 440B. In some embodiments, the combined length of the extendable sizing adapter portion length 401 of the first extendable sizing adapter portion 440A and the extendable sizing adapter portion length of the second extendable sizing adapter portion 440B does not exceed the adapter body length 405.

In some embodiments, the first extendable sizing adapter portion 440A is confined to a first portion of the medical tube adapter having an adapter body first portion length of approximately half of the adapter body length 405. In some embodiments, a first extended body length 402 corresponds to the first extendable sizing adapter portion 440A being in an extended position. The first extended body length 402 may be about 70 mm to about 100 mm. In some embodiments, the second extendable sizing adapter portion 440B is confined to a second portion of the medical tube adapter having an adapter body first portion length of approximately half of the adapter body length 405. In some embodiments, a second extended body length corresponds to the second extendable sizing adapter portion 440B being in an extended position. The second extended body length may be substantially similar to the first extended body length 402.

The second port 420 may have a port height 403, which is a diameter of the second port 420 from one or more outer edges of the second port 420. In some embodiments, the port height 403 is about 18 mm to about 26 mm. The first port 410 may have a port height substantially similar to the port height 403. In some embodiments, the first port 410 has a port height different from the port height 403. The third port 430 may have a port diameter 406 measured from one or more outer edges of the third port 430. In some embodiments, the port diameter 406 is about 21 mm to about 29 mm.

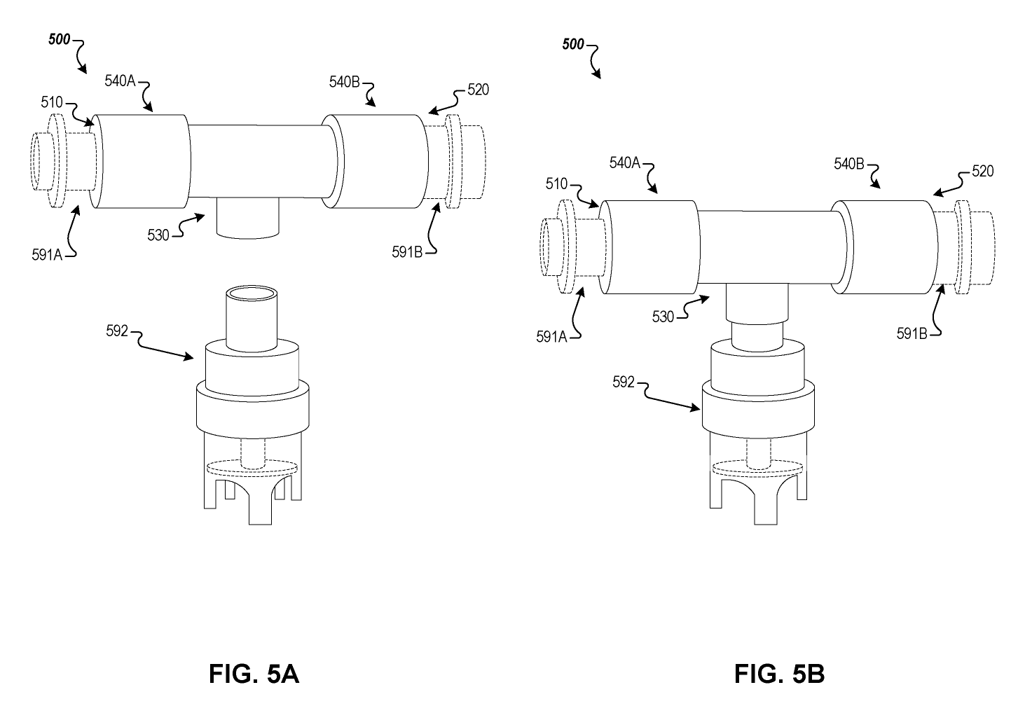

The second extendable sizing adapter portion 440B may have an adapter height 404, which is a diameter of the second extendable sizing adapter portion 440B from one or more outer edges of the second extendable sizing adapter portion 440B. In some embodiments, the adapter height 404 is about 25 mm to about 33 mm. The first extendable sizing adapter portion 440A may have an adapter height substantially similar to the adapter height 404. In some embodiments, the first extendable sizing adapter portion 440A has an adapter height different from the adapter height 404.FIGS. 5A-8B illustrate connectable medical tube adapters including various adapters, according to some embodiments. Components and features of FIGS. 1-2, 3A-3E, 4A-4E, 5A-5B, 6A-6B, 7A-7B, 8A, and/or 8B that have the same or similar name and/or reference number may have the same or similar functionality, material, components, etc.

FIGS. 5A-B illustrate connectable medical tube adapters 500, according to some embodiments. The connectable medical tube adapter 500 may include a first port 510 configured to receive (e.g., connect with) a first adapter 591A, a second port 520 configured to receive a second adapter 591B, and/or a third port 530 configured to receive an adapter 592.

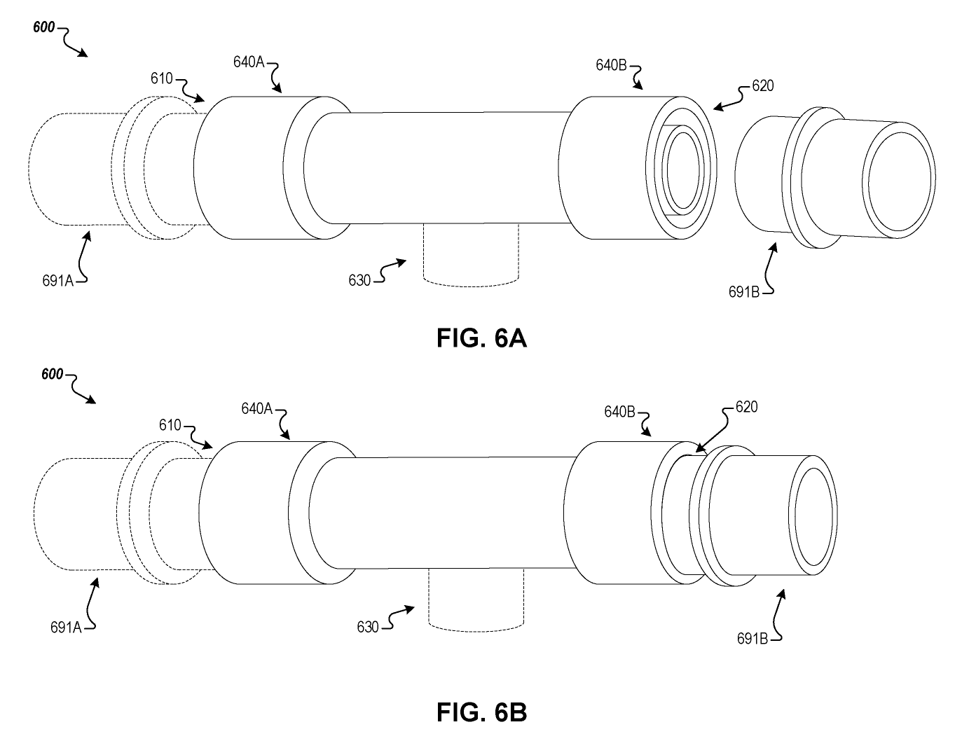

FIGS. 6A-B illustrate connectable medical tube adapters 600, according to some embodiments. The connectable medical tube adapter 600 may include a first port 610 configured to receive a first adapter 691A and/or a second port 620 configured to receive a second adapter 691B. In some embodiments, the connectable medical tube adapter 600 includes a third port 630. The third port 630 may be configured to receive an adapter (e.g., the adapter 592 of FIGS. 5A-B) or another type of adapter.

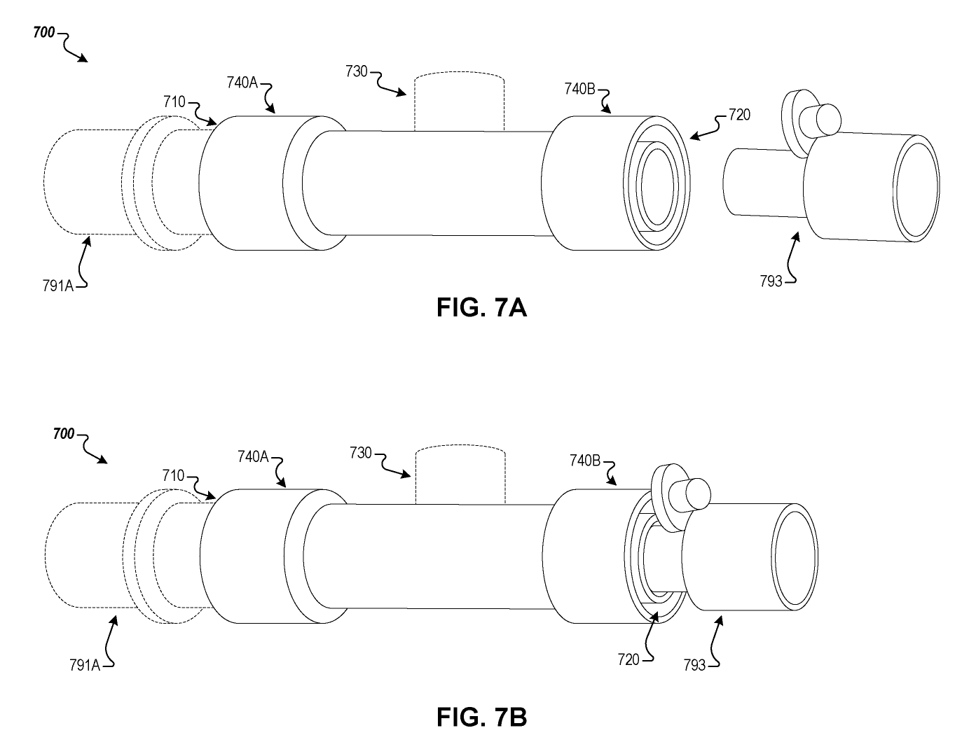

FIGS. 7A-B illustrate connectable medical tube adapters 700, according to some embodiments. The connectable medical tube adapter 700 may include a first port 710 configured to receive a first adapter 791A and/or a second port 720 configured to receive a sensor 793. The sensor 793 may be one of a flow sensor, a pressure sensor, an air-in-line (e.g., bubble) sensor, a temperature sensor, a blockage sensor, a chemical sensor, an optical sensor, a captive and/or proximity sensor, and/or a position and/or motion sensor. In some embodiments, the connectable medical tube adapter 700 includes a third port 730. The third port 730 may be configured to receive an adapter (e.g., the adapter 592 of FIGS. 5A-B) or another type of adapter.

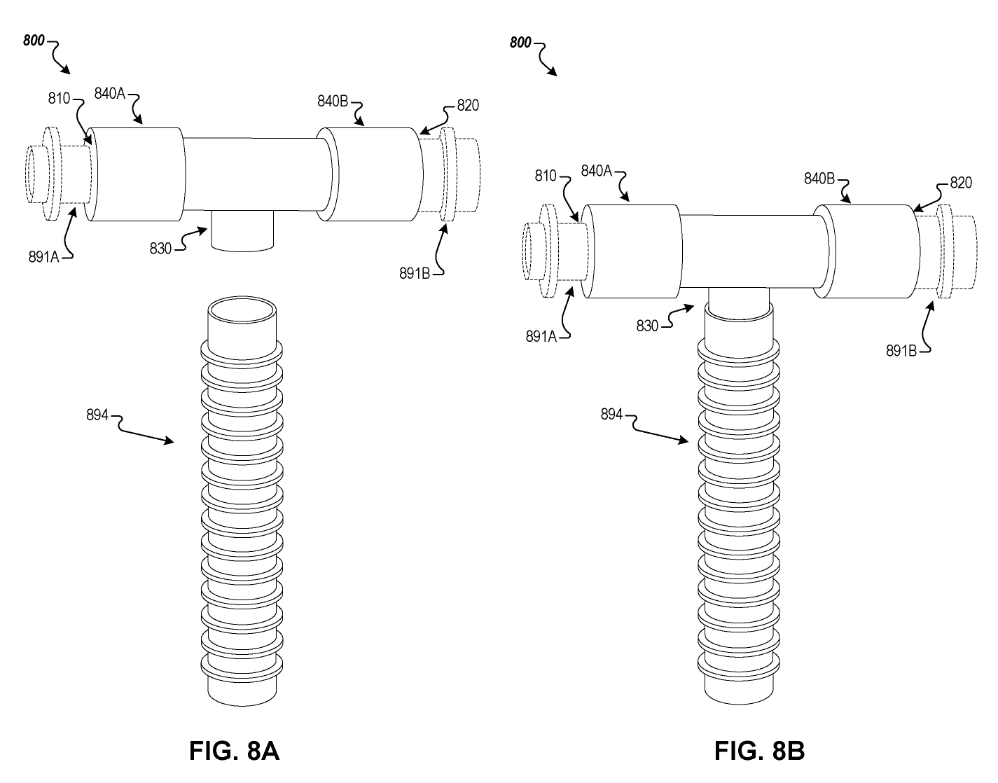

FIGS. 8A-B illustrate connectable medical tube adapters 800, according to some embodiments. The connectable medical tube adapter 800 may include a first port 810 configured to receive a first adapter 891A, a second port 820 configured to receive a second adapter 891B, and/or a third port 830 configured to receive a hose 894. In some embodiments, the third port 830 is configured to receive other types of adapters.

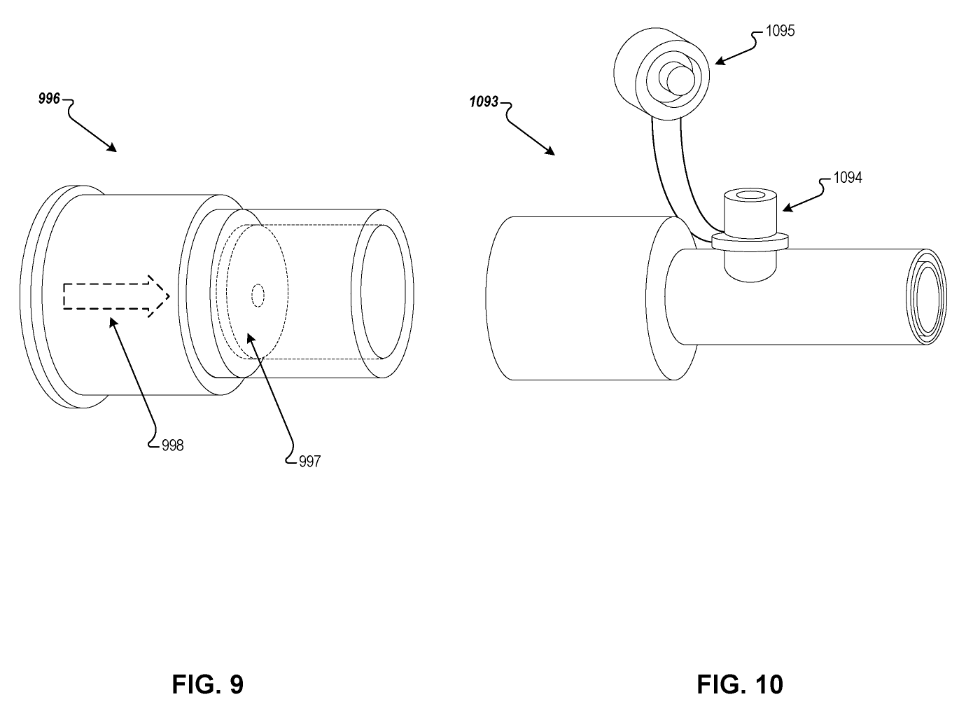

FIG. 9 illustrates a one-way valve adapter 996 (e.g., a check valve), according to some embodiments. The one-way valve adapter 996 may be configured to allow fluid to flow in a single direction (e.g., prevents fluid from flowing backward, backflowing, etc.). The one-way valve adapter 996 may include a valve 997 configured to open when pressure is applied in the direction of the fluid flow 998 and close when pressure is applied in a direction other than the fluid flow 998. The one-way valve adapter 996 may be connected to a medical tube adapter (e.g., the medical tube adapters of FIGS. 1, 2, 3A-E, 4A-E, 5A-B, 6A-B, 7A-B, and/or 8A-B) via a port (e.g., the first port 210, the second port 220, and/or the third port 230 of FIG. 2).

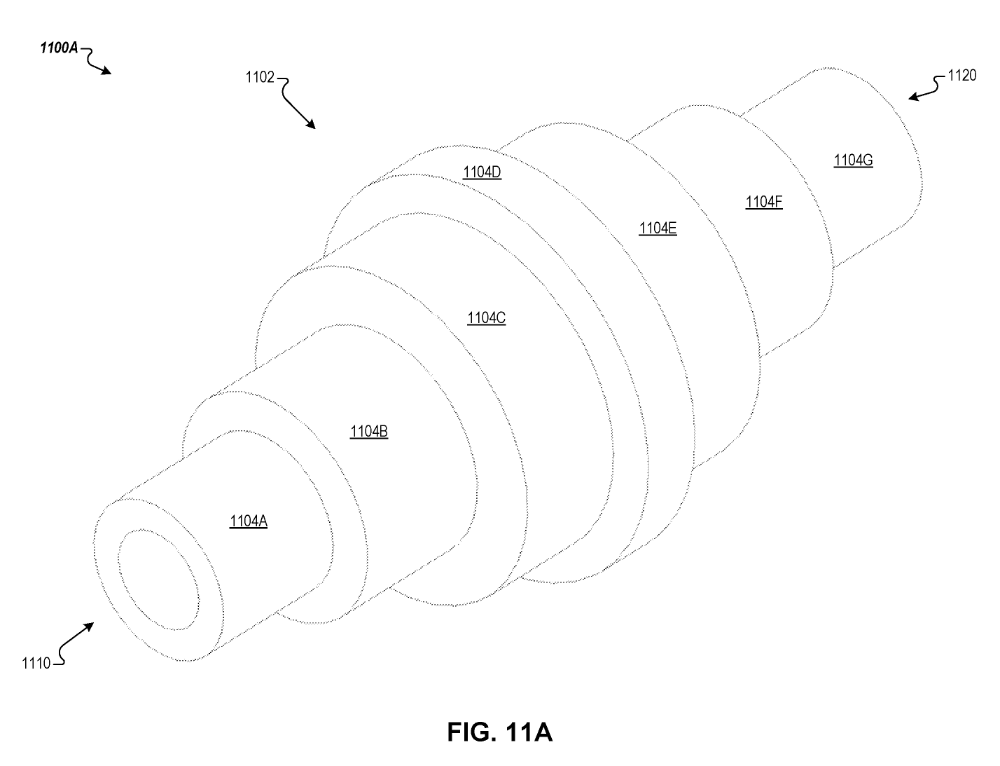

FIG. 10 illustrates a sensor adapter 1093, according to some embodiments. The sensor (e.g., of sensor adapter 1093) may be one of a flow sensor, a pressure sensor, an air-in-line (e.g., bubble) sensor, a temperature sensor, a blockage sensor, a chemical sensor, an optical sensor, a captive and/or proximity sensor, and/or a position and/or motion sensor. In some embodiments, the sensor adapter 1093 includes a sensor port 1094 configured to receive smaller hoses, tubes, and/or other components. For example, the sensor port 1094 may be used to inject (e.g., add) medicine into an existing fluid flow. In another example, the sensor port 1094 may be used to insert a probe to measure gas levels, temperatures, flow rates, etc. In some embodiments, when the sensor port 1094 is not in use, the sensor port cap 1095 may cover the sensor port 1094 to prevent fluid from escaping the sensor adapter 1093.

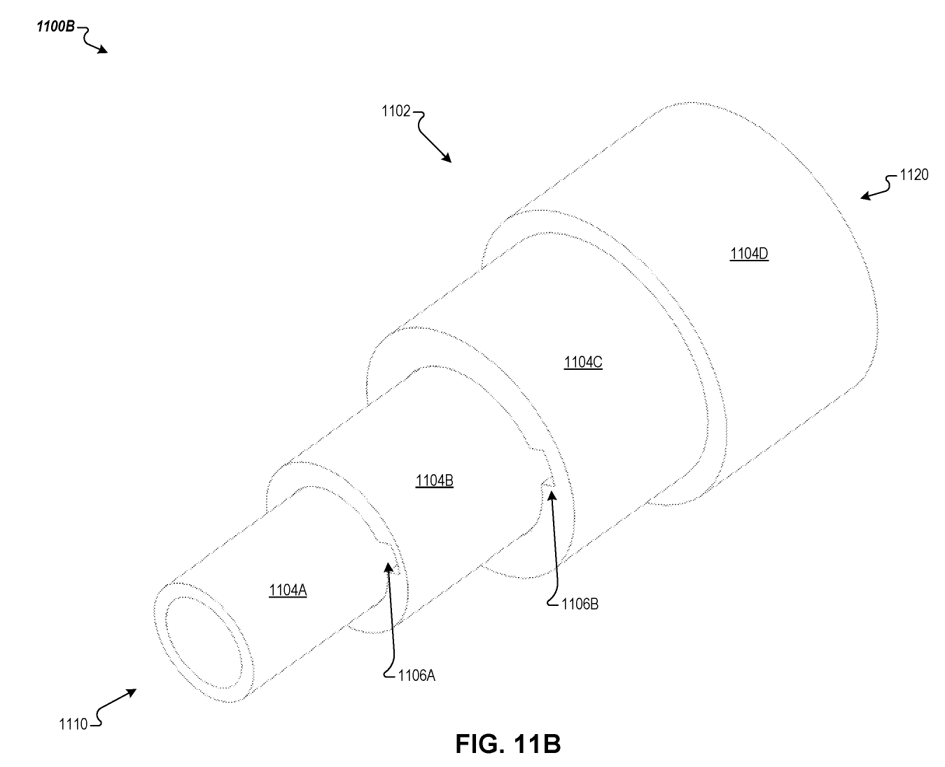

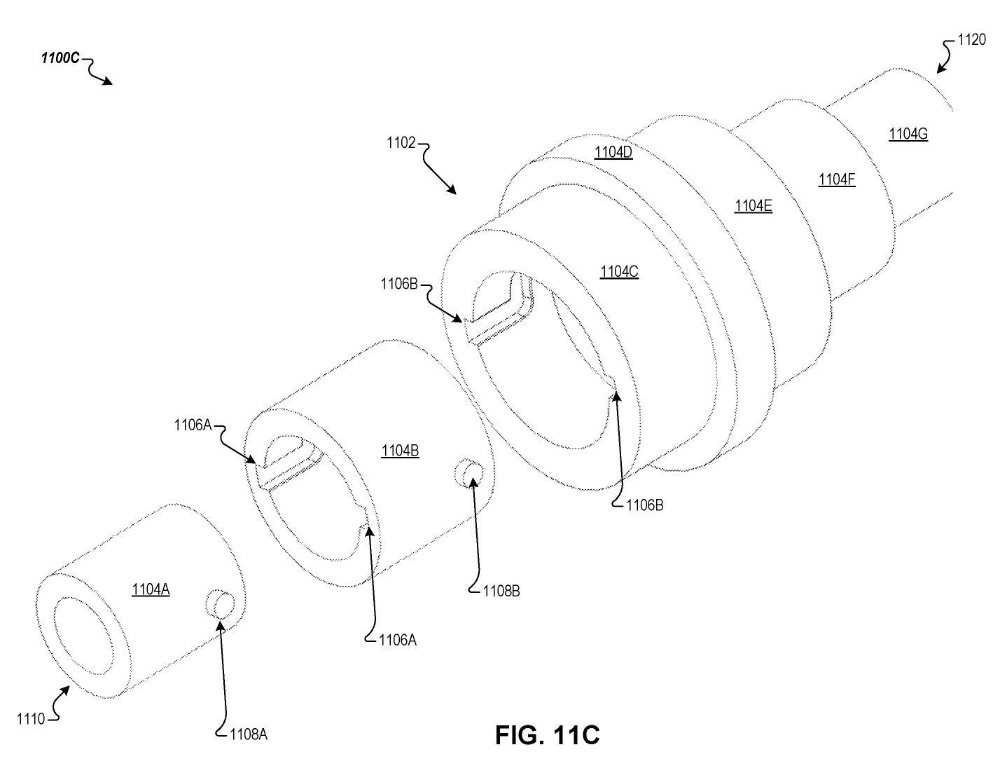

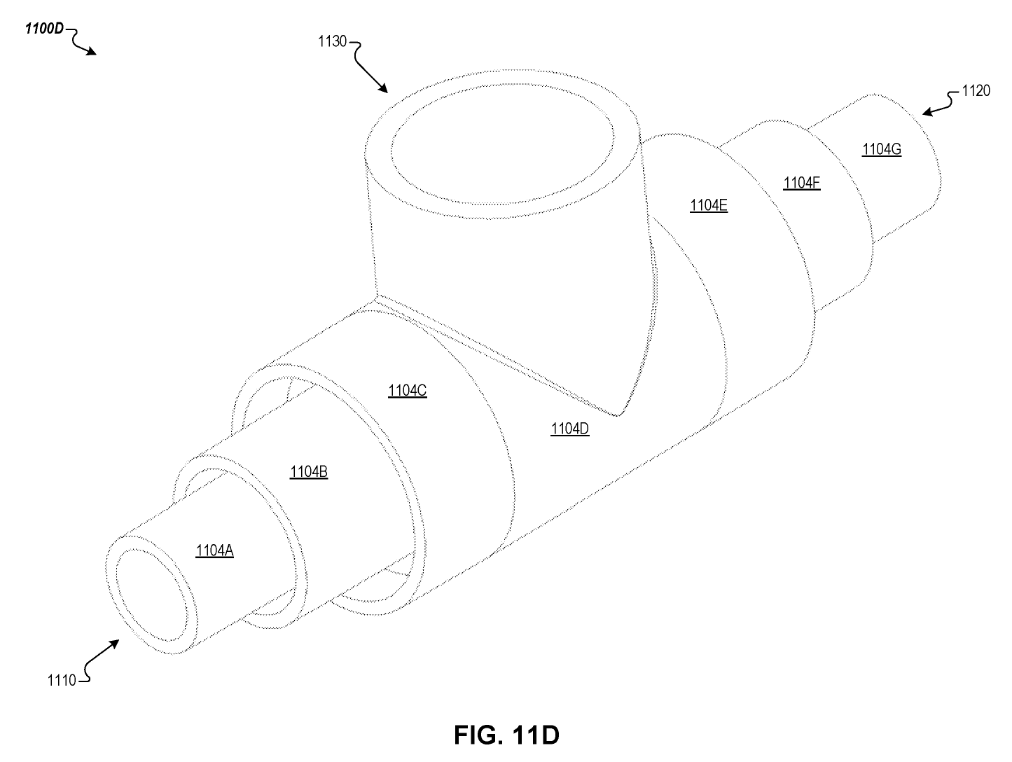

FIGS. 11A-D illustrate medical tube adapters 1100A-D, according to some embodiments. A medical tube adapter 1100 may include one or more ports and each port may be configured to connect to one or more sizes of tubing. A port may be a stepped port where a distal end of the port is configured to connect to one or more different sizes of tubing, a first intermediate portion of the port is configured to connect to one or more different sizes of tubing, and/or a second intermediate portion of the port is configured to connect one or more different sizes of tubing.

The medical tube adapter 1100A may include a body 1102. Medical tube adapter 1100A may include one or more portions 1104(e.g., 1104A-G). Each of the portions may protrude to a different location and may have one or more surfaces that are configured to connect to a different size of tubing. Each of the portions 1104 may be located around substantially the same axis (e.g., a center of each portion 1104 may be along the same axis).

Referring to FIG. 11A, medical tube adapter 1100A may have stepped portions 1104 and one or more of the portions 1104 may be configured to connect to one or more different sizes of tubing.

The portion 1104D may have a largest diameter of the portions 1104. In some embodiments, tubing that connects to portion 1104C and/or 1104E is to abut a sidewall of portion 1104D. In some embodiments, tubing is to connect to an outer surface of portion 1104D.

Portion 1104C may extend from portion 1104D in a first direction and portion 1104E may extend from portion 1104D in a second direction that is opposite the first direction. In some embodiments, portion 1104C and portion 1104E both have the same outer diameter. In some embodiments, tubing is to connect to an outer surface of portion 1104C and/or tubing is to connect to an outer surface of portion 1104E. In some embodiments, tubing that connects to an outer surface of portion 1104B is to abut a sidewall of portion 1104C and/or tubing that connects to an outer surface of portion 1104F is to abut a sidewall of portion 1104E.

Portion 1104B may extend from portion 1104C in a first direction and portion 1104F may extend from portion 1104E in a second direction that is opposite the first direction. In some embodiments, portion 1104B and portion 1104F both have the same outer diameter. In some embodiments, tubing is to connect to an outer surface of portion 1104B and/or tubing is to connect to an outer surface of portion 1104F. In some embodiments, tubing that connects to an outer surface of portion 1104A is to abut a sidewall of portion 1104B and/or tubing that connects to an outer surface of portion 1104G is to abut a sidewall of portion 1104F.

Portion 1104A may extend from portion 1104B in a first direction and portion 1104G may extend from portion 1104F in a second direction that is opposite the first direction. In some embodiments, portion 1104A and portion 1104G both have the same outer diameter and/or a same inner diameter. In some embodiments, tubing is to connect to an outer surface of portion 1104A, tubing is to connect to an inner surface of portion 1104A, tubing is to connect to an outer surface of portion 1104F, and/or tubing is to connect to an inner surface of portion 1104F.

In some embodiments, a medical tube adapter 1100 may have less or more portions 1104 than those shown in one or more of FIGS. 11A-11D.

In some embodiments, the portions 1104A-1104G are integral to each other. In some embodiments, the portions 1104A-1104G are non-removably fixed to each other. In some embodiments, one or more of the portions 1104A-1104G are removably connected to each other.

Referring to FIG. 11B, medical tube adapter 1100B may include portions 1104A and 1104B that are removable from other portions of the medical tube adapter 1100B. The medical tube adapter 1100B may include a body 1102 that includes portions 1104C and/or 1104D. Portion 1104B may have one or more protrusions 1108B extending from an outer surface that interface with one or more recesses 1106B formed by an inner surface of portion 1104C. Portion 1104A may have one or more protrusions 1108A extending from an outer surface that interface with one or more recesses 1106A formed by an inner surface of portion 1104B. In some embodiments, portion 1104A may be removed to connect tubing to portion 1104B. In some embodiments, portions 1104A-B may be removed to connect tubing to portion 1104C.

Although the second port 1120 is not shown in FIG. 11B, the second port 1120 may be substantially similar in structure and functionality to the first port 1110.

Referring to FIG. 11C, medical tube adapter 1100C may include portions 1104A, 1104B, 1104F, and/or 1104G that are removable from other portions of the medical tube adapter 1100C. The medical tube adapter 1100C may include a body 1102 that includes portions 1104C, 1104D, and/or 1104E. Portion 1104B may have one or more protrusions 1108B extending from an outer surface that interface with one or more recesses 1106B formed by an inner surface of portion 1104C. Portion 1104A may have one or more protrusions 1108A extending from an outer surface that interface with one or more recesses 1106A formed by an inner surface of portion 1104B. In some embodiments, portion 1104A may be removed to connect tubing to portion 1104B. In some embodiments, portions 1104A-B may be removed to connect tubing to portion 1104C. Portions 1104E-G may be similar in structure, removability, functionality, size, etc. to portions 1104C-A. In some embodiments, portions 1104E-G may be an exact mirror to portions 1104C-A. In some embodiments, portions 1104E-G may have one or more differences (e.g., different size) compared to portions 1104C-A.

Referring to FIG. 11D, medical tube adapter 1100D may include portions 1104A-C and 1104E-G that include a lip, where each of portions 1104A-C and 1104E-G can each connect to two sizes of tubing.

In some embodiments, a first size of tubing can connect to an inner surface of portion 1104A, a second size of tubing can connect to an outer surface of portion 1104A, a third size of tubing can connect to an inner surface of portion 1104B, a fourth size of tubing can connect to an outer surface of portion 1104B, a fifth size of tubing can connect to an inner surface of portion 1104C, and a sixth size of tubing can connect to an outer surface of portion 1104C.

Portions 1104E-G may be similar to (e.g., an exact mirror of) Portions 1104A-C.

Medical tube adapter 1100D may include a body that includes port 1110 (e.g., portions 1104A-C), port 1120 (e.g., portions 1104E-G), and port 1130 (e.g., extends from or is part of portion 1104D). Ports 1110 and 1120 may be disposed along a first central axis. Port 1130 may have a central axis that is substantially perpendicular to the first central axis.

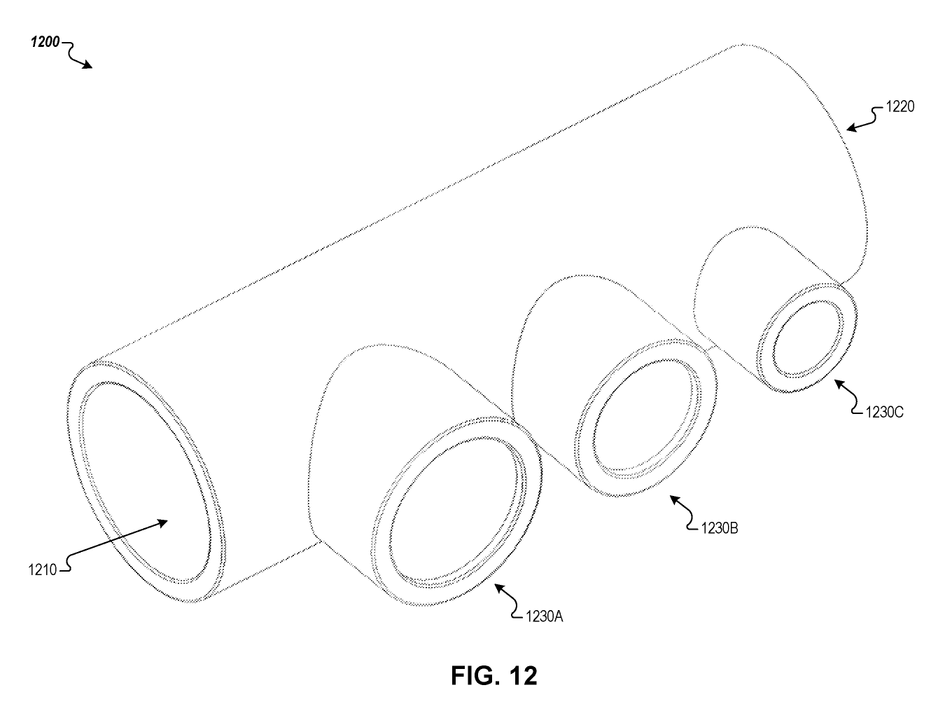

FIG. 12 illustrates a medical tube adapter 1200, according to some embodiments. Medical tube adapter 1200 may have a first port 1210 and a second port 1220 that are centrally disposed along a first axis. Medial tube adapter 1200 may have one or more additional ports 1230 (e.g., ports 1230A-C) that each have a central axis that is substantially perpendicular to the first axis. Each of the one or more additional ports 1230 may be configured to connect to one or more different sizes of tubing compared to each other. For example, port 1230A may be configured to connect to a first size of tubing (on an outer surface of port 1230A) and/or a second size of tubing (on an inner surface of port 1230A, port 1230B may be configured to connect to a third size of tubing (on an outer surface of port 1230B) and/or a fourth size of tubing (on an inner surface of port 1230B), and/or port 1230C may be configured to connect to a fifth size of tubing (on an outer surface of port 1230C) and/or a sixth size of tubing (on an inner surface of port 1230C).

In some embodiments, instead of a port 1220, the distal end of medical tube adapter 1200 is capped.

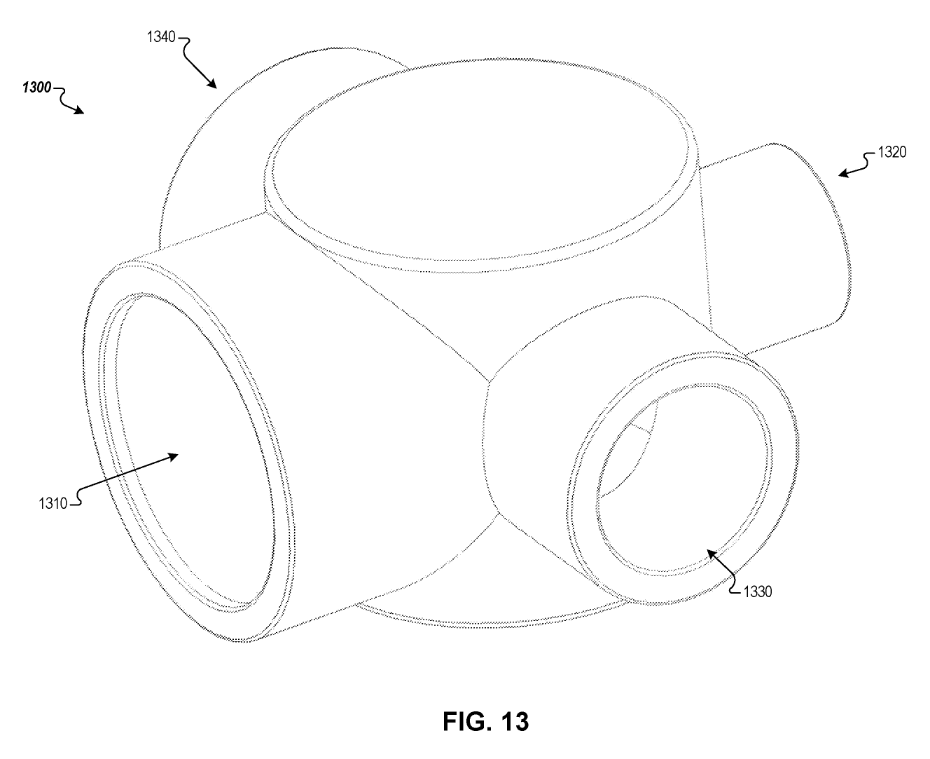

FIG. 13 illustrates a medical tube adapter 1300, according to some embodiments. Medical tube adapter 1300 include a first port 1310 that extends in a first direction and a second port 1320 that extends in a second direction that is substantially opposite the first direction. The first port 1310 and the second port 1320 may be disposed along a first axis (e.g., have flow paths centered on a first axis).

Medical tube adapter 1300 may include a third port 1330 that extends in a third direction and a fourth port 1340 that extends in a fourth direction that is substantially opposite the third direction. The third port 1330 and the fourth port 1340 may be disposed along a second axis (e.g., have flow paths centered on a second axis) that is substantially orthogonal to the first axis.

One or more of medical tube adapters of FIGS. 1-13 may have one or more of the same functionalities, structures, materials, etc. In some embodiments, a medical tube adapter may have one or more features of two or more of FIGS. 1-13 (e.g., features from multiple FIGS. may be combined together). In some embodiments, a medical tube adapter may have less features than those shown in any one or more of FIGS. 1-13.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

In some embodiments, the terms "first," "second," "third," "fourth," etc. as used herein are meant as labels to distinguish among different elements and do not have an ordinal meaning according to their numerical designation.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A, X includes B, or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ± 5%, 10%, 15%, 20%, 25%, 30%, 35%, 40%, 45%, 50%, 55%, 60%, 65%, 70%, 75%, 80%, 85%, 90%, 95%, 100%, 105%, and/or 110%.

For simplicity of explanation, methods herein are depicted and described as a series of acts or operations. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A medical tube adapter comprising:
a first port configured to connect to at least three different sizes of tubing; and
a second port.

2. The medical tube adapter of claim 1, wherein the first port is configured to connect to at least four different sizes of tubing.

3. The medical tube adapter of claim 2, wherein the first port comprises:
an inner cylindrical lip comprising a first inner surface configured to connect to a first size of tubing and a first outer surface configured to connect to a second size of tubing; and
an outer cylindrical lip comprising a second inner surface configured to connect to a third size of tubing and a second outer surface configured to connect to a fourth size of tubing.

4. The medical tube adapter of claim 1, wherein the second port is configured to connect to at least three corresponding different sizes of tubing.

5. The medical tube adapter of claim 4, wherein the second port comprises:
an inner cylindrical lip comprising a first inner surface configured to connect to a first size of tubing and a first outer surface configured to connect to a second size of tubing; and
an outer cylindrical lip comprising a second inner surface configured to connect to a third size of tubing and a second outer surface configured to connect to a fourth size of tubing.

6. The medical tube adapter of claim 5, wherein the second port further comprises
an extendable sizing adapter comprising:
an extendable sizing adapter inner surface configured to slidably interface with the second outer surface, the extendable sizing adapter inner surface configured to connect to a fifth size of tubing responsive to extending beyond the outer cylindrical lip; and
an extendable sizing adapter outer surface configured to connect to a sixth size of tubing.

7. The medical tube adapter of claim 6, wherein the extendable sizing adapter further comprises:
a first extendable sizing adapter portion configured to slidably interface with the medical tube adapter in a first direction; and
a second extendable sizing adapter portion configured to slidably interface with the medical tube adapter in a second direction.

8. The medical tube adapter of claim 1 further comprising a third port that is configured to connect to at least two different sizes of tubing.

9. The medical tube adapter of claim 8, wherein the third port comprises:
a cylindrical lip comprising a first inner surface configured to connect to a first size of tubing; and
an outer surface configured to connect to a second size of tubing.

10. The medical tube adapter of claim 8 further comprising a substantially airtight component configured to prevent fluid flow through the third port.

11. The medical tube adapter of claim 10, wherein the substantially airtight component comprises a cap or a plug.

12. The medical tube adapter of claim 1, wherein the a first port comprises:
a first portion configured to connect to a first size of tubing of the at least three different sizes of tubing;
a second portion that is configured to removably connect to the first portion and configured to connect to a second size of tubing of the at least three different sizes of tubing; and
a third portion that is configured to removably connect to the second portion and configured to connect to a third size of tubing of the at least three different sizes of tubing.

13. The medical tube adapter of claim 12, wherein the first removable portion comprises one or more outer protrusions configured to interface with one or more inner recesses of the second removable portion.

14. A medical tube adapter comprising:
a body comprising a first port configured to connect to at least a first size of tubing and a second size of tubing; and
an extendable sizing adapter movably disposed around the first port, wherein the extendable sizing adapter is configured to connect to at least a third size of tubing and a forth size of tubing, wherein the first size of tubing, the second size of tubing, the third size of tubing, and the forth size of tubing are different sizes from each other.

15. The medical tube adapter of claim 14, wherein a distal outer portion of the first port has a first outer diameter, wherein a distal portion of the extendable sizing adapter that overlaps with the first port has a first inner diameter that is less than the first outer diameter to prevent the extendable sizing adapter from sliding off of the first port.

16. The medical tube adapter of claim 14, wherein the extendable sizing adapter comprises a first portion and a second portion that secure together around the first port via one or more protrusions and one or more recesses.

17. A medical tube adapter comprising:
a body comprising a first port, a second port, and a third port;
a first extendable sizing adapter movably disposed around the first port, wherein the first extendable sizing adapter is configured to connect to at least a first size of tubing and a second size of tubing,; and
a second extendable sizing adapter movably disposed around the second port, wherein the second extendable sizing adapter is configured to connect to at least a third size of tubing and a forth size of tubing, wherein the first size of tubing, the second size of tubing, the third size of tubing, and the forth size of tubing are different sizes from each other.

18. The medical tube adapter of claim 17, wherein the first port and the second port are substantially co-axial, and wherein the third port is substantially orthogonal to the first port and the second port.

19. The medical tube adapter of claim 17, wherein the first port is configured to connect to at least three different sizes of tubing.

20. The medical tube adapter of claim 19, wherein the second port is configured to connect to at least three corresponding different sizes of tubing.
